(19) United States
(12) Patent Application Publication
(10) Pub. No.: US 2026/0102731 A1
(43) Pub. Date: Apr. 16, 2026

(54) PRODUCING GAS MIXTURES OF HYDROGEN AND CARBON DIOXIDE

(71) Applicant: Wacker Chemie AG, Munich (DE)

(72) Inventors: Torsten GOTTSCHALK-DAUDIG, München (DE); Jochen DAUTH, München (DE); Sebastian KRÖNER, München (DE); Marcus SCHÄFER, München (DE)

(73) Assignee: Wacker Chemie AG, Munich (DE)

(21) Appl. No.: 19/117,096

(22) PCT Filed: Oct. 12, 2022
(86) PCT No.: PCT/EP2022/078378

§ 371 Date: Mar. 31, 2025

(51) Int. Cl. B01D 53/04; C07C 29/151

(52) U.S. Cl. CPC B01D 53/0438; C07C 29/1518; B01D 2257/504; B01D 2259/4009

(57) ABSTRACT

A process for producing a hydrogen-carbon dioxide gas includes isolating CO₂ from gas mixtures having a volume fraction of CO₂ in the range from 0.01% to 20% using an adsorber and desorption of the CO₂ using a heated hydrogen-containing gas stream in a desorber. The process also includes adjusting the CO₂ volume fraction of the hydrogen-containing gas stream to the target content using a cycle gas system. The heated hydrogen-CO₂ gas mixture is completely or partially passed through one or more desorbers thereby increasing the CO₂ volume fraction in the hydrogen-CO₂ gas mixture until achievement of a target value.

The invention relates to a process for obtaining a hydrogen-carbon dioxide gas mixture and the use thereof for producing methanol.

### PRIOR ART

The isolation of CO₂ from process offgases is an important aspect for climate-neutral industrial production of raw materials or energy. The prior art employs so-called amine scrubbing where CO₂ is passed through an aqueous amine solution and is reversibly isolated from the gas stream through carbamine formation. Disadvantages of this technology include inter alia that effective separation of the CO₂ requires a relatively high CO₂ partial pressure. That is to say that this process is not suitable for process offgases having a low CO₂ content or isolation of CO₂ directly from the atmosphere (direct air capture; DAC). This problem can be solved by adsorption of CO₂ on solid adsorbers. The problem here is the desorption of CO₂ from the adsorber. This requires markedly increasing the temperature in the adsorber in a temperature swing process for example. The introduction of superheated steam for example is typical here. This means that the CO₂ requires energy-intensive drying after desorption. It would be better to have a gas mixture that is amenable to further processing directly after desorption without costly and inconvenient treatment. The invention proposed here makes this possible. The use of hydrogen gas as desorber gas and simultaneously as heat-transfer medium makes it possible to efficiently obtain a hydrogen-carbon dioxide gas mixture which is subsequently employable for production of methanol for example without costly and complex treatment.

The use of hydrogen for desorption of carbon dioxide is not new. Thus, DE 10 2013 022 021 B4 claims a process for isolating CO₂ from gas mixtures where the CO₂-rich gas mixture is passed through a gas chromatography column and the CO₂ diffuses physically more slowly than the tail gases. After loading of the column, said column is ISOTHERMALLY backwashed with hydrogen, i.e. the column is not heated during the desorption step. This is disadvantageous especially in the case of chemical adsorbers since desorption is an endothermic process and desorption is effected only incompletely, if at all, without temperature elevation.

EP 3530640 A discloses a process for reductive production of methane from CO₂ and hydrogen. CO₂ is desorbed from the adsorber with preheated hydrogen and the hydrogen is heated by the waste heat from the reduction reactor. The desorbed H₂/CO₂ mixture is also admixed with fresh hydrogen to establish the necessary H₂/CO₂ ratio. The disadvantage is that in the case of a low-CO₂ gas mixture there is no way to increase the CO₂ partial pressure of the gas mixture.

It is accordingly an object of the present invention to overcome the disadvantages of the prior art and in particular to find a simple and cost-effective way to adapt the CO₂ content in a hydrogen-carbon dioxide gas mixture within wide limits.

### DESCRIPTION OF THE INVENTION

The present invention provides a process for producing a hydrogen-carbon dioxide gas mixture comprising the following process steps:
I) isolation of CO₂ from gas mixtures having a volume fraction of CO₂ in the range from 0.01% to 20% using an adsorber;
II) desorption of the CO₂ using a heated hydrogen-containing gas stream in a desorber; and
III) adjustment of the CO₂ volume fraction of the hydrogen-containing gas stream to the target content using a cycle gas system, by means of which the heated hydrogen-CO₂ gas mixture is passed through the desorber, thus increasing the CO₂ volume fraction in the hydrogen-CO₂ gas mixture.

### Description of the Individual Process Steps

### I) Isolation:

The carbon dioxide is preferably isolated from process offgases or other industrial or natural gas mixtures. The gas mixtures preferably have a CO₂ volume fraction in the range from 0.01% to 20%, in particular 0.04% to 15%. It is preferable when the gas mixture is purified of harmful constituents such as reactive secondary gases such as sulfur dioxide, nitrogen oxides and/or finely divided solids such as dusts before the adsorption step. This may be done using established technologies such as wet scrubbers, deNOx systems (catalytic nitrogen oxide reduction systems, nitrogen oxide removal systems), electrofilters, cyclones and dust filters etc. The adsorption is preferably carried out in the manner of a chemisorption, i.e. a chemical reaction between the carbon dioxide and the adsorber occurs during the adsorption. The moisture content of the gas mixture is therefore adapted before entry into the adsorber. The optimal moisture content of the gas mixture depends on the chemical reaction in the chemisorption step. The isolation of the carbon dioxide is preferably carried out in adsorbers provided with solid adsorbents. The adsorption step may be carried out in a gas-borne or stirred fluidized bed or in a fixed bed, wherein preference is given to a fixed bed adsorber. The number of adsorbers is preferably between 2 and 10, especially between 2 and 5. The adsorption temperature is preferably in the range of 0-150° C., particularly preferably between 20-100° C., i.e. the process gas temperature upon entry into the adsorber is preferably in the range of 0-150° C., particularly preferably between 20-100° C. The pressure in the adsorber is preferably in the range of 0-50 bar (absolute), particularly preferably in the range of 0.5-10 bar (absolute).

### II) Desorption:

It is preferable when the CO₂-laden adsorber is then separated from the CO₂-rich process gas stream and connected as a desorber. The desorber is thus an adsorber in regeneration. Desorption employs a preheated hydrogen-rich gas. The hydrogen volume fraction in the desorber gas may be in the range from 100% to 0.1%. It is thus possible to employ pure hydrogen or a gas mixture, wherein the second gas component is carbon dioxide having a volume fraction of 99.1% to 0%. Passage through the desorber increases the CO₂ content in the desorber gas. This especially applies to a partial pressure-independent adsorption and desorption. That is to say the capacity is defined solely/primarily by the temperature conditions, such as is typically the case for chemisorption. Once physisorption plays a relevant role, high CO₂ contents in the desorption gas are detrimental or require overcompensation through further temperature increases. In addition to heating via the CO₂-rich process gas the adsorber may be heated for example via a heating jacket, for example using heat-transfer liquids or gases, or be electrically heated from outside. The adsorber may optionally also be heated via heatable internals in the adsorber such as heating coils, heating plates or heating fingers.

The heating of the CO₂-rich process gas is preferably carried out via a gas heater such as for example plate heat exchangers, shell-and-tube heat exchangers, finned heaters, heating registers or electric resistance heaters. Suitable energy sources for the operation of the gas heater include electrical energy (resistance heating) or fluid heat transfer media such as thermal oil, hot water/pressurized water and steam, preferably from thermal recovery systems. Energy couplings for thermal recovery are possible when heat is generated at a temperature at least 20 K higher than the adsorption temperature in processes in spatial proximity, for example heat flows from combustion plants upstream of the CO₂ recovery plant or else downstream process steps for CO₂ utilization (for example exothermic methanol synthesis, optional process step IV).

### III) Adjustment of the CO₂ Volume Fraction:

The inventive adjustment of the CO₂ volume fraction is preferably carried out via a cycle gas circuit, by means of which the desorber gas is completely or partially passed through one or more desorbers until the CO₂ content in the desorber gas achieves the target value which is required for example for a subsequent process step, for example the methanol synthesis. In a first inventive embodiment (IIIa) the cycle gas circuit is configured such that the desorber emitting the cycle gas is identical to the desorber receiving the cycle gas. In an inventive operating mode of the cycle gas system the CO₂-laden adsorber is purged with cold fresh hydrogen in a first step to displace residual amounts of the process offgas from the adsorber. This purge gas amount may either be discarded or recycled into the CO₂-rich process gas stream. Once the purity of the purge gas downstream of the absorber has reached a value of preferably at least 99% by volume, in particular at least 99.5% by volume, the desorption step begins through commencement of the cycle gas process. To this end the adsorber to be regenerated (which thus becomes a desorber) is purged with heated cycle gas. The cycle gas may be pure hydrogen or a hydrogen-carbon dioxide gas mixture. The cycle gas is cycled through the desorber until the gas composition corresponds at least to the target value, i.e. at least the target value of the CO₂ volume fraction. Once the target value has been reached, at least one substream is withdrawn from the cycle gas and optionally sent for further processing, for example reductive methanol synthesis. The subamount withdrawn from the cycle gas is preferably replaced by addition of pure hydrogen into the cycle gas circuit. The desorption step is terminated when the CO₂ content of the cycle gas no longer substantially changes during passage through the desorber. The cycle gas process is then terminated and residual amounts of the cycle gas in the desorber are subsequently displaced with cold hydrogen gas or process gas, preferably process gas, while simultaneously cooling the desorber. This purge gas amount may either be discarded or supplied to the cycle gas stream or the process gas stream. Once the CO₂ content in the purge gas is less than or equal to the CO₂ content in the process gas and/or the temperature in the desorber is less than or equal to the process gas temperature before entry into the adsorber, the purging step is terminated and the desorber may be used as an adsorber again.

In a particularly preferred embodiment the process according to the invention comprises at least two independent cycle gas systems for producing a hydrogen-carbon dioxide gas mixture in order to allow continuous operation of the subsequent optional utilization of the gas mixture, for example the reductive methanol synthesis.

In a further embodiment according to the invention (IIIb) the desorber emitting the cycle gas and the desorber receiving the cycle gas are at least partially distinct, i.e. the desorber completely laden with CO₂ is connected to a cycle gas process in which at least one further desorber is already in the desorption process, wherein the desorbers may be connected in series or parallel, preferably in parallel. Analogously to embodiment IIIa the connection of the desorber completely laden with CO₂ is carried out after displacement of the process gas residual amount by purging with fresh hydrogen or cycle gas, preferably cycle gas. The purge gas amount may either be discarded or recycled into the CO₂-rich process gas stream. Once the purity of the purge gas downstream of the absorber in the case of fresh hydrogen as purge gas has achieved a value of at least 99% by volume, in particular at least 99.5% by volume, or in the case of cycle gas as purge gas has achieved the composition of the cycle gas, the desorption step is begun through commencement of the cycle gas process. To this end the adsorber to be regenerated (which thus becomes a desorber) is purged with heated cycle gas. The cycle gas may be pure hydrogen or a hydrogen-carbon dioxide gas mixture. The cycle gas is cycled through the desorbers until the gas composition corresponds at least to the target value, i.e. the target value of the CO₂ volume fraction. Once the target value has been reached, at least one substream is withdrawn from the cycle gas and optionally sent for further processing, for example reductive methanol synthesis. The subamount withdrawn from the cycle gas is preferably replaced by addition of pure hydrogen into the cycle gas circuit. The desorption step of at least one desorber is terminated when the CO₂ content of the cycle gas no longer substantially changes during passage through the desorber. The corresponding desorber(s) are separated from the cycle gas system. Residues of the cycle gas in the desorber are subsequently displaced with cold hydrogen gas or process gas, preferably process gas, while simultaneously cooling the desorber. This purge gas amount may either be discarded or supplied to the cycle gas stream or the process gas stream. Once the CO₂ content in the purge gas is less than or equal to the CO₂ content in the process gas and/or the temperature in the desorber is less than or equal to the process gas temperature before entry into the adsorber the purging step is terminated and the desorber may be used as an adsorber again.

The aforementioned cycle gas systems preferably comprise not only suitable pipe conduits with connection options to the different adsorbers/desorbers but also suitable ventilators/compressors for conveying the gases and suitable heating systems for adjusting the required temperatures of the cycle gas. A cycle gas system is preferably also equipped with suitable feed and discharge systems for gases and measurement and control technology, for example CO₂ concentration measurement instruments (e.g. mass spectrometry, process gas chromatography, Raman spectroscopy) and conventional measurement instruments for pressure, temperature and flow rates, for controlled process management.

The temperature of the desorber gas is preferably in the range of 20-250° C., particularly preferably in the range of 50-200° C., and the pressure is preferably in the range of 0-50 bar (absolute), particularly preferably 0.5-10 bar (absolute). The temperature of the desorber gas in step II is preferably at least 5° C. higher, particularly preferably at least 10° C. higher, in particular at least 20° C. higher, than the temperature of the gas mixture in step I. In addition to the heating by the desorber gas the desorber may be heated for example via a heating jacket, for example by heat-transfer liquids or gases, or electrically from outside. The desorber may optionally also be heated via heatable internals in the desorber such as heating coils, heating plates or heating fingers.

The heating of the desorber gas is preferably carried out via a gas heater such as for example plate heat exchangers, shell-and-tube heat exchangers, finned heaters, heating registers or electrical resistance heaters. The gas heater is preferably employed in the cycle gas system to allow repeated readjustment to the desired value of the entry temperature of the desorber gas into the desorber. Suitable energy sources for the operation of the gas heater include electrical energy (resistance heating) or fluid heat transfer media such as thermal oil, hot water/pressurized water and steam, preferably from thermal recovery systems. Energy couplings for thermal recovery are possible when heat is generated at a temperature at least 20 K higher than the desorption temperature in processes in spatial proximity, for example heat flows from combustion plants upstream of the CO₂ recovery plant or else downstream process steps for CO₂ utilization (for example exothermic methanol synthesis, optional process step IV).

It is preferable when the number of desorbers corresponds to the number of adsorbers. This is to be understood as meaning that while one adsorber is active another adsorber whose absorption capacity for CO₂ has been reached is regenerated by purging with desorber gas and thus serves as a desorber during this time. This is to be understood as meaning that the process according to the invention preferably comprises at least two adsorbers. However, for reasons of the total capacity of the process it is advantageous to increase the number of adsorbers. The number of adsorbers is preferably in the range of 2-10, particularly preferably in the range of 2-5. This is to be understood as meaning that at least one adsorber is active, at least one adsorber is subjected to regeneration and thus serves as a desorber and the remaining number of adsorbers may be maintained in standby mode in the regenerated state, i.e. without CO₂ loading.

In a preferred embodiment, process step III is followed by the use of the hydrogen-carbon dioxide gas mixture withdrawn from the cycle gas circuit for production of methanol. In a preferred embodiment the hydrogen-carbon dioxide gas mixture may be admixed with further fresh hydrogen after withdrawal from the cycle gas circuit and before introduction into the methanol production process especially in order, in the case of an excessively high CO₂ volume fraction of the hydrogen-carbon dioxide gas mixture, to allow adjustment thereof to the precise target value for the methanol synthesis.

### IV) Optional Use of the Hydrogen-Carbon Dioxide Gas Mixture:

The gas mixture having an adjusted carbon dioxide volume fraction may subsequently be used for producing methanol in the manner of a synthesis gas. It is preferable when the proportion of hydrogen in this gas mixture is 75 mol %, i.e. the proportion of CO₂ is 25 mol %. The reduction of the CO₂ by the hydrogen in the gas mixture is carried out in reactors of established design (e.g. catalytic fixed bed reactors) using suitable catalysts. The hydrogen-carbon dioxide gas mixture optionally requires preheating to the necessary reaction temperature before reaction. The precise temperature and pressure depend on the selected catalyst. Since the reduction of CO₂ with hydrogen to afford methanol is exothermic at −49.6 kJ/mol (at 300 K) the heat of reaction liberated thereby may be employed, for example using heat exchangers, to heat the hydrogen-carbon dioxide gas mixture or else to heat the desorber gas to liberate the carbon dioxide in desorption step II.

1-8. (canceled)

9. A process for producing a hydrogen-carbon dioxide gas mixture comprising the following process steps:
I) isolation of CO₂ from gas mixtures having a volume fraction of CO₂ in the range from 0.01% to 20% using an adsorber;
II) desorption of the CO₂ using a heated hydrogen-containing gas stream in a desorber; and
III) adjustment of the CO₂ volume fraction of the hydrogen-containing gas stream to the target content using a cycle gas system, by means of which the heated hydrogen-CO₂ gas mixture is completely or partially passed through one or more desorbers, thus increasing the CO₂ volume fraction in the hydrogen-CO₂ gas mixture, until achievement of a target value.

10. The process as claimed in claim 9, wherein in step I the gas mixtures preferably have a CO₂ volume fraction in the range from 0.04% to 15%.

11. The process as claimed in claim 9, wherein in step I the adsorber is a fixed bed adsorber.

12. The process as claimed in claim 9, wherein the adsorber laden with CO₂ in step I is separated from the CO₂-rich process gas stream and connected as a desorber in step II.

13. The process as claimed in claim 9, wherein the temperature of the desorber gas in step II is at least 5° C. higher than the temperature of the gas mixture in step I.

14. The process as claimed claim 9, wherein in step III the cycle gas system is configured such that the desorber emitting the cycle gas is identical to the desorber receiving the cycle gas.

15. The process as claimed in claim 9, wherein in step III the desorber emitting the cycle gas and the desorber receiving the cycle gas are distinct, wherein the desorber completely laden with CO₂ is connected to a cycle gas process in which at least one further desorber is already in the desorption process.

16. The process as claimed in claim 9, wherein process step III is followed by a process step IV in which after adjustment of the required CO₂ volume fraction the hydrogen-carbon dioxide gas mixture is used for production of methanol.
